(19) United States
(12) Patent Application Publication
(10) Pub. No.: US 2011/0091973 A1
(43) Pub. Date: Apr. 21, 2011

(54) Modified and fusion enhanced erythrocytes, cells and uses thereof

(76) Inventor: Larry F. Glaser, Fairfax Station, VA (US)

(21) Appl. No.: 12/314,341

(22) Filed: Dec. 9, 2008

(51) Int. Cl. C12N 15/63; C12N 5/10

(52) U.S. Cl. 435/455; 435/325

(57) ABSTRACT

Modified fusion enhanced erythrocytes (or other cell types and synthetic cells) including human viral receptor proteins, human viral coreceptor proteins and viral derived proteins capable of mediating entry of respective viruses into the modified erythrocytes, cells or pseudo-cells and the method of using the fusion enhanced modified erythrocytes, cells or pseudo-cells for the treatment or prevention of viral infections. The fusion enhanced modified erythrocytes comprises CD4 and at least one HIV coreceptor, such as CXCR4 or CCR5 and as well, at least one of cholesterol rafts, fusin, actin, a viral derived protein such as fusion peptide derived from HIV GP120 or HIV GP41 or a shorter protein derived from a long viral protein, such as a portion of HIV derived GP120, or HIV GP41 such as the 23 N-terminal peptide of the HIV-1 gp 41 protein (AVGIGALFLGFLGAAGSTMGARS) called FP23 (Fusion Peptide). These viral-fusion enhanced cells may also be electrostatic charge enhanced through further additions named in this invention. The modified erythrocytes, when administered to an HIV patient, bind to the plasma virus and induce the injection of the HIV ribonucleoprotein complex into the cells. The entrapped viral content is sequestered within said cell for at least the period of time that the cell maintains its outer membrane integrity. The virus is thereafter either degraded or deactivated within the erythrocytes, cells or pseudo-cells, or destroyed by erythrophagocytosis.

### TECHNICAL FIELD

The present invention relates to the creation of novel viral traps in the form of cells or pseudo-cells equipped with exogenous proteins and lipids or, equipped with concentrations of endogenous proteins and lipids in specific concentrations not found within the requisite cell type or combinations of exogenous proteins and endogenous proteins. The present invention proffers and defines fusion enhanced modified erythrocytes including enucleated erythrocytes, fusion enhanced and modified cells and methods of using the same for the treatment and prevention of viral infections.

### BACKGROUND

Human immunodeficiency virus (HIV) infection is characterized as a systemic immunosuppressive disorder caused by the viral-mediated depletion of CD4 T cells or viral mediated loss of immune competence, which develops into the profound immunodeficiency that underlies the acquired immunodeficiency syndrome (AIDS). AIDS is characterized by various pathological conditions, including immune incompetence, opportunistic infections, neurological dysfunctions, and neoplastic growth.

Many drugs have been approved for the treatment of AIDS. Non-limiting examples of these drugs include nonnucleoside reverse transcriptase inhibitors, such as delavirdine (Rescriptor, Pfizer), Efavirenz (Sustiva, Bristol-Myers Squibb), and evirapine (Viramune, Boehringer Ingelheim); nucleoside reverse transcriptase inhibitors, such as Abacavir (Ziagen or ABC, GlaxoSmithKline), Didanosine (Videx or ddl, Bristol-Myers Squibb), Emtricitabine (Emtriva, Gilead Sciences), Lamivudine (Epivir, GlaxoSmithKline), Stavudine (Zerit, Bristol-Myers Squibb), Tenofovir DF (Viread, Gilead Sciences), Zalcitabine (Hivid, Hoffman-La Roche), Zidovudine (Retrovir or AZT, GlaxoSmithKline); protease inhibitors, such as Amprenavir (Agenerase, GlaxoSmithKline and Vertex Pharmaceuticals), Atazanavir (Reyataz, Bristol-Myers Squibb), Fosamprenavir (Lexiva, GlaxoSmithKline and Vertex Pharmaceuticals), Indinavir (Crixivan, Merck), Lopinavir (Kaletra, Abbott Laboratories), Nelfinavir (Viracept or NFV, Agouron Pharmaceuticals), Ritonavir (Norvir or RTV, Abbott Laboratories), Saquinavir (Fortovase, Hoffman-La Roche); and fusion inhibitors, such as Enfuvirtide (Fuzeon, Hoffman-La Roche and Trimeris).

The recommended treatment for HIV is a combination of three or more medications in a regimen called “highly active antiretroviral therapy” or “HAART.” Exemplary HAART regimens include Sustiva+Epivir+(Retrovir, Viread or Zerit), Kaletra+Epivir+(Retrovir or Zerit), Sustiva+Emtriva+(Retrovir or Viread or Zerit), Kaletra+Emtriva+(Retrovir or Zerit), or Reyataz+(Epivir or Emtriva)+(Retrovir or Zerit). Introduction of HAART have led to a dramatic decline in both HIV-related illness and death. Early clinical trials demonstrated a reduction of plasma HIV RNA loads to undetectable levels in the majority of treated individuals. Subsequent studies, however, showed more limited success in achieving and maintaining viral suppression. Many patients experienced immunologic and clinical responses to HAART without sustained suppression of plasma viremia. Therefore, significant challenges still remain in the scientific and clinical battle against HIV and AIDS. In particular, there is a need for new methods that can effectively reduce plasma viremia in HIV-infected individuals.

### SUMMARY OF THE INVENTION

The present invention addresses this need by providing modified erythrocytes and other cell types which comprise HIV receptors and fusion enhancers capable of mediating HIV entry into the modified cells. These modified erythrocytes and other cell types, when administered to an HIV+ patient, absorb and entrap plasma HIV, preventing the virus from infecting native CD4⁺ lymphocytes. The entrapped viral content is either degraded or deactivated within the erythrocytes, or is sequestered for the duration of entrapment and ultimately destroyed by erythrophagocytosis. The present invention also features modified erythrocytes or other cell types which comprise receptor proteins and fusion enhancers for other viruses, and methods of using these erythrocytes for the treatment or prevention of other viral infections. As aforementioned, the present invention features non-erythrocyte cells capable of capturing and internalizing viruses. This can include any cell or cell-like artifice taken from or modified from any source, including mammals. In all examples, it is important to note the net sum effect of sequestering viral particles from reaching any and all other cell types. The hallmarks of the invention include the recognition that viral particles in mammals have short half lives. Movement into the cells of this invention sequesters the viral particles such that time elapses and the particles become non-infectious by simple passage of time. Further, the uncoating of the virion or the chemistry change of environments from outside a cell to inside, places each particle in a state where there is no potential for movement to a new cell. Placement of a viral particle in a mature red blood cell introduces an unanticipated chemistry to the viral content. The particle can be further disabled aside from these aforementioned aspects, through contact with the elements within the cell of this invention. In an enucleated erythrocyte, the natural chemistry of the red cell will trigger HIV to start its RT function. Given the specific conditions within a mature red cell, including but not limited to ph, lack of nucleus, lack of ribosomes, lack of organelles, presence of cutting enzymes and other features of the cell, HIV will start but will not progress through its RT cycle, the initial replication stage post entry into a new host cell. As such, it is further anticipated there will be a damage caused to the HIV RNA backbone (twin RNAs) which is not repairable by the viral content and as such, the HIV remnants will be rendered non-infectious should by some chance thereafter, escape the sequestering effect of the cell. Lastly, there is mention of the use of further content contained within the cells of this invention, to further assure the sequestering of each viral particle within is further met with a disablement mechanism that is permanent with respect to disabling the viral particle content. Those of skill recognize these potential elements, which can be loaded into the Red Blood Cell (RBC). HAART components, hammer head ribozymes, siRNAs and the like, would all serve as requisite examples, however, another goal would be to use that which does not in any way, affect RBC function.

In one aspect and embodiment, the present invention features a modified erythrocyte which comprises fusion enhancement proteins or nucleotides and a recombinantly-produced receptor protein capable of binding to a virus. As used herein, “recombinantly produced” means that the receptor protein, or its coding sequence (including 5′ or 3′ regulatory regions), is prepared or modified using recombinant DNA technology. It is also noted, cell loading techniques can be utilized to produce the requisite cells, or to further modify cells produced with recombinant technology, in a multi-stage strategy for producing the cells.

In one embodiment, the recombinantly-produced receptor protein comprises an extracellular domain of a CD4 protein. As a non-limiting example, the recombinantly-produced receptor protein comprises or consists of a human CD4 protein. Human fusin is another embodiment and example of a receptor protein which can function to move a virus, such as HIV, from outside a cell to inside a cell, operating as a sole receptor but also known to operate more efficiently in the presence of other classes of co-receptor proteins. Integrin alpha-4 beta-7 is yet another candidate as a cellular receptor for HIV virus, used in similar context for purpose of this invention. With this filing, the use of fusion enhancers for each modality, is disclosed.

X-ray crystallography has thus far revealed two structural classes of fusion glycoprotein (Kielian, 2006↓; Kielian & Rey, 2006↓; Skehel & Wiley, 2000↓; Stiasny & Heinz, 2006↓). Class I fusion proteins [e.g. human immunodeficiency virus 1 (HIV-1)gp41 FP-23, influenza virus HA2] are identified as occurring within helical, trimeric rods that project as spikes from the viral envelope. In the fusion-activated state, their N (fusion peptide-proximal) and C (TMD-proximal) termini become juxtaposed at one end of a helical hairpin core domain. Class II fusion glycoproteins (e.g. flavivirus E, alpha virus E1) comprise three domains rich in β-strands that lie roughly parallel to the viral membrane. At neutral pH, the metastable state of E, which has dual receptor-binding and fusion functions, is maintained in a homodimer by monomer-monomer interactions that sequester the fusion loop. In the case of alphaviruses, glycoprotein E2 mediates receptor binding, whereas the associated E1 trimer mediates fusion. E1 metastability is maintained through E1-E2 interactions. At low fusion pH, E and E1 have almost identical trimeric structures where membrane-inserted fusion loops are atop three uptilted protomers. Trimerization creates three surface-exposed hydrophobic grooves along the trimer axis for the antiparallel packing of the TMD-proximal amphipathic α-helical stem to form a hairpin. Thus, hairpin formation is employed by both classes of fusion glycoprotein to appose membrane-associated fusion peptides and TMDs, which leads to membrane fusion. These factors are important as they delineate how viruses, which carry water molecules on their outermost extensions, overcome hydrophobic localized repulsion found between virus and cell. A cell loaded with viral glycoprotein fusion fragments will exhibit more capacity to fuse to viral particles and internalize the particles at a greater rate and with more reliability. It is thus an embodiment of the present invention to incorporate viral fusion proteins at various stages of cell production to yield cells which do not occur in nature. Rather than the target virus providing the catalytic fusion peptide, we provide said peptide sequence in advance of the virus' arrival. As a non-limiting example, HIV fusion peptide and Hepatitis C fusion peptide could be utilized to load a cell intended to be used in a viral trap strategy, as an HIV preventative or therapeutic. As such, we have not limited the invention to using the same class of receptor/coreceptor or fusion enhancer and fusion peptide sequence focused on only one viral strain or clade as the source, meaning, we can use HIV receptor/coreceptor and fusion peptide taken from Hepatitis C if we wish. Any one viral fusion peptide may find utility in enhancing viral fusion for a cell intended to fuse with a completely different viral strain, hence the need to be clear that we intend to allow this crossing under the control of the manufacturing processes. It is anticipated that fusion enhancement derived from a specific virus, such as using HIV related fusion peptide sequences, will function efficiently with HIV human viral receptors and coreceptors. However, it is also anticipated that fusion enhancement derived from one virus, such as Hepatitis C, will also offer fertile ground for cross utilization with HIV human viral receptors and coreceptors as human viruses utilize superfamilies of proteins which in some combinations traverse the viral species or clades, and offer function such as in this case, serving to catalyze the initial fusion reaction of virus particle to a cell membrane. Specific reference to the 23 N-terminal peptide of the HIV-1 gp 41 protein (AVGIGALFLGFLGAAGSTMGARS) called FP23 is drawn and incorporated here. Any and all fragments drawn from any and all mammalian viruses, taken from the glycoprotein complex of each virus, eludicated as viral protein fragments, are claimed herein as useful to prime the receptor coreceptors of this invention and further catalyze fusion to virions and internalization of virion content within the cells of this invention. Nothing herein is intended to limit the use of any viral protein fragment or residue, taken from one viral strain or clade and used to predispose a given receptor coreceptor class to allow for more efficient fusion of virion particles. Simply stated, we could prime an HIV receptor/coreceptor of this invention with HIV derived residues or, find a Hepatitis C residue that is useful and prime with that residue individually or in combination with HIV derived residues and others.

In another embodiment, the recombinantly-produced receptor protein comprises an extracellular domain of an HIV coreceptor. Examples of HIV coreceptors suitable for the present invention include, but are not limited to, CXCR4, CCR5, CCR1, CCR2, CCR3, CCR4, CCR8, CXCR1, CXCR2, CXCR3, CXCR6, GPR15, APJ, CMKLR1, or CX3CR1. In a specific example, the recombinantly-produced receptor protein comprises or consists of an HIV coreceptor selected from CXCR4 or CCR5.

In still another embodiment, a modified erythrocyte of the present invention comprises CD4 or Integrin alpha-4 beta-7, Fusin or both and at least one HIV coreceptor, e.g., CXCR4, CCR5, CCR1, CCR2, CCR3, CCR4, CCR8, CXCR1, CXCR2, CXCR3, CXCR6, GPR15, APJ, CMKLR1, or CX3CR1. In one example, the modified erythrocyte comprises CD4 and an HIV coreceptor selected from CXCR4 or CCR5. In another example, the modified erythrocyte comprises CD4, Fusin, CXCR4, and CCR5.

In each embodiment herein, fusion enhancers are added to the cells. Said addition may be performed by recombinant technology, or through any cell loading technique including but not limited to ghosting (chemical methods), electro-insertion (electroporation), spinoculation (exerting limited centripetal or centrifugal forces to merge fusion enhancers into the cell membrane) or through creation of multimeric (oligomers) units. Fusion enhancers include cholesterol rafts, actin, fusin, viral derived fusion peptide and viral derived proteins. HIV Fusion peptide FP-23 is a requisite example of a fusion enhancer derived from a virus. FP-23 is also a requisite example of a short viral protein fragment derived from HIV GP41.

Prior to use of any cell loading technique to manufacture the cells of this invention, human derived viral receptor proteins, such as CD4 and Fusin, and a human derived viral coreceptor proteins, such as CCR5, may be premixed in a suitable medium to allow for bonding between the receptor coreceptor proteins. In this mix cholesterol rafts, actin, fusin and viral derived proteins may be included. Said mix can be prepared according to standard laboratory procedure utilized for cell loading, leaving the proteins functional, post loading. The order of, and concentration of proteins and cholesterol into this mix will be variable within set limits with receptor, coreceptor and viral derived proteins provided in generally equal amounts and cholesterol rafts provided at 0.001% up to 5% of the molecular weight of the mixed components. One reason for variability allowing a net positive result is the fact that any unused protein or lipid not bound to the cell, is removed in a final wash process. These skills are known to the art of cell loading, electroinsertion and electroporation, cell ghosting and thus need not be repeated here. The purpose is to allow interaction of the named components which are proteins derived from human cells and viruses, and one named fat (cholesterol or cholesterol raft) prior to attempting to attach the oligomers to a cell utilizing cell loading rather than stem cell recombinant and natural growth (colony expansion), as a technique to arrive at the same net sum cell with its new function of fusion enhanced highly targeted viral binding capacity. Cell loading provides for en masse modification of cells and provides more diversity than recombinant technology because one can treat en masse, several sub classes of cell in the same one effort. Recombinant growth from stem cells yields less diversity of cell sub types. Recombinant technology also yields cells with very specific occurrences of receptor/coreceptors while loading allows one to literally dial select the receptor/coreceptor occurrences within reasonable, logical limits. Suffice to say what a recombinant cell offers in terms of receptor/coreceptor occurrences per cell, can be matched with cell loading or demonstrated at concentration levels of 2-10,000 fold more occurrences per cell. The logical limits are those where a cell, overloaded with receptor/coreceptors cause any negative side effect which the host cannot tolerate, or, where the cell has other functions we would like to leave in tact and thus we need to scale the receptor/coreceptor occurrences to leave other endogenous cell functions in a more productive state, operating at normal capacity.

The modified erythrocytes of the present invention can be prepared from erythrocyte precursor cells, such as hematopoietic progenitor cells. Erythrocyte precursor cells can be isolated from peripheral blood, bone marrow, umbilical cord blood, or other suitable sources. Expression vectors encoding desired receptor proteins can be introduced into these precursor cells by transfection, transduction, electroporation, gene gun, or other gene transfer techniques. Alternatively, the endogenous genes that encode the desired receptor proteins can be modified to increase their transcription/translation activities. Precursor cells thus modified can be cultured under erythropoiesis conditions to generate terminally-differentiated, enucleated erythrocytes that express the desired receptor proteins.

The present invention also contemplates the use of other methods for preparing erythrocytes of the present invention. For instance, viral receptor proteins can be incorporated into mature enucleated erythrocytes through membrane fusion or other suitable means, as appreciated by those of ordinary skill in the art. As a non-limiting example, liposomes or micelles comprising desired viral receptor proteins (e.g., CD4, CXCR4, CCR5, or other HIV coreceptors) can be prepared using conventional techniques and then fused with mature enucleated erythrocytes. Mature enucleated erythrocytes thus modified can be administered to individuals in need thereof for the treatment or prevention of viral infections. Preferably, the donor of the mature erythrocytes is also the recipient of the modified cells.

In another aspect, the present invention features cell samples comprising modified erythrocytes of the present invention. A cell sample of the present invention can have a volume of from 10 to 1,000 ml, such as 50, 100, 200, 300, 400, 500, 600, 700, 800, or 900 ml. Each sample can include at least 1×10¹⁰, 1×10¹¹, 1×10¹², 1×10¹³, or more erythrocytes of the present invention.

In yet another embodiment of the invention, for all cells produced by these teachings, static charge enhancement per cell, is proposed. Additives are disclosed which will increase the static charge, particularly for a mobile cell, such as the RBC. Aside from naturally found metals and metal oxides, I propose non-toxic biodegradable polymers as additives to cells, to increase their charge to increased limits which pose no harm to the biological systems of the host. The purpose is to increase the frequency of the initial bond to a targeted virus, which is an electrostatic bond.

### DESCRIPTION OF THE INVENTION

The present invention features methods for treating or preventing viral infections (e.g., HIV infections). These methods typically comprise administering a plurality of erythrocytes of the present invention to an individual in need thereof. In one example, the individual being treated has contracted HIV or is at risk of HIV contraction. The erythrocytes being administered comprise CD4 and at least one HIV coreceptor, such as CXCR4 or CCR5. Preferably, the erythrocytes being administered have the same ABO blood type as that of the recipient. More preferably, the erythrocytes are prepared from hematopoietic progenitor cells isolated from the recipient. In another example, the modified erythrocytes are prepared from mature enucleated erythrocytes isolated from the recipient. In many cases, the erythrocytes employed are modified with CD4 and HIV coreceptor(s) which are identical to the recipient's endogenous proteins.

The present invention further features the use of non-erythrocyte cells for the treatment or prevention of viral infections. The nuclei of these cells can be deactivated by radiation, chemical treatment, or other suitable means. These cells comprise the receptor protein(s) capable of mediating entry of a virus of interest into the cells. In one embodiment, the non-erythrocytes cells of the present invention are leukocytes which comprise CD4 and at least one HIV coreceptor (e.g., CXCR4 or CCR5). In many cases, the non-erythrocytes cells are modified with CD4 and HIV coreceptor(s) which are identical to the recipient's endogenous proteins.

Other features, objects, and advantages of the present invention are apparent in the detailed description that follows. It should be understood, however, that the detailed description, while indicating preferred embodiments of the invention, is given by way of illustration only, not limitation. Various changes and modifications within the scope of the invention will become apparent to those skilled in the art from the detailed description.

The present invention features modified erythrocytes which comprise receptor proteins for HIV or other viruses. These receptor proteins can mediate entry of the respective viruses into the modified cells, thereby removing the viruses from the blood or other tissues that are accessible by the erythrocytes. Because erythrocyte lacks nucleic acid synthesis machinery, an entrapped virus cannot replicate or otherwise initiate viral functions. As a result, the entrapped virus is either degraded or deactivated within the erythrocytes, or destroyed by phagocytes during erythrophagocytosis. Non-erythrocytes are also provided which can entrap the virus and prevent its use in cells which would otherwise serve the virus as a valid host cell, where the non-erythrocyte cannot serve as a host cell for the replication of the virus as caused by modifications to the cell as described herein.

The modified erythrocytes of the present invention can be prepared from hematopoietic progenitor cells transfected or transduced with exogenous genes that encode desired viral receptor proteins. Exemplary procedures suitable for this purpose are described in Malik et al., Blood, 91:2664-2671 (1998); Hanspal et al., Blood, 84:3494-3504 (1994); Wada et al., Blood, 75:505-511 (1990); and Fibach et al., Blood, 73:100-103 (1989), all of which are incorporated herein by reference in their entireties. In one example, hematopoietic progenitor cells are isolated from peripheral blood, bone marrow, or umbilical cord blood. These cells are typically CD34 positive and, therefore, can be purified using immunomagnetic beads coupled with anti-CD34 antibodies. The purified progenitor cells are transfected or transduced with expression vectors that encode viral receptor proteins, and then cultured under erythroid differentiation conditions (e.g., high concentrations of erythropoietin (EPO) and low concentrations of granulocyte-macrophage colony-stimulating factor (GM-CSF) and IL-3) to produce terminally-differentiated, enucleated erythrocytes that express the viral receptor proteins. Erythrocytes thus prepared are negative for DNA staining and therefore can be separated from other cells in the culture by using cell sorting techniques such as flow cytometers or fluorescence activated cell sorters.

In one aspect, the present invention features modified erythrocytes comprising HIV receptors. HIV is a member of the lentivirus family of retroviruses. There are two prevalent types of HIV, HIV-1 and HIV-2. Various strains having been identified for each type of HIV. HIV uses a receptor-mediated pathway in the infection of host cells. HIV-1 requires contact with two cell-surface receptors to gain entry into cells and initiate infection. CD4 is the primary receptor. CXCR4 and CCR5, members of the chemokine receptor family of proteins, serve as secondary coreceptors for HIV-1 strains that are tropic for T-cell lines or macrophages, respectively. Many HIV-2 strains also utilize CCR5 or CXCR4 to enter host cells.

CD4 (CD 4 antigen (p55)) is a cell-surface glycoprotein found on the mature helper T cells and immature thymocytes, as well as on monocytes and macrophages. Some cytotoxic T cells and natural killer cells also express CD4 protein. An exemplary human CD4 sequence is depicted in SEQ ID NO:1.

CCR5 (chemokine (C—C motif) receptor 5) is a member of the beta chemokine receptor family, which is predicted to have seven transmembrane domains similar to G protein-coupled receptors. This protein is expressed by T cells and macrophages, and is known to be a co-receptor for macrophage-tropic virus, including HIV, to enter host cells. Defective alleles of this gene have been associated with the HIV infection resistance. Expression of CCR5 was also detected in a promyeloblastic cell line. An exemplary human CCR5 sequence is illustrated in SEQ ID NO:2.

CXCR4 (chemokine (C—X—C motif) receptor 4; also known as fusin) is a CXC chemokine receptor specific for stromal cell-derived factor-1. CXCR4 also has seven transmembrane regions. It acts with the CD4 protein to support HIV entry into cells. Alternate transcriptional splice variants encoding different CXCR4 isoforms have been identified. Two exemplary CXCR4 isoforms are depicted in SEQ ID NOs: 3 and 4, respectively.

Without limiting the present invention to any particular theory, it is believed that the interaction between the viral envelope glycoprotein gp120/gp41 and CD4 triggers the fusion between viral and host membranes. This interaction, which is also facilitated by cell surface glycosaminoglycans, leads to conformational changes in gp120, which results in the interaction between gp120 and a secondary coreceptor, mostly CCR5 or CXCR4. The double engagement of CD4 and a secondary coreceptor induces a sharp conformational change of a second viral envelope protein, gp41, which acts as a fusogenic component leading to the fusion of viral and cell membranes required for the injection of the HIV ribonucleoprotein complex into the host cell cytoplasm. This invention seeks to leverage the interaction of any viral protein which forms catalytic reactions with the cell receptor/coreceptor protein complex that can be isolated and identified, sourced to a specific viral residue and leveraged for use as a fusion enhancer motif.

It has been reported that HIV-1 strains transmitted in vivo generally use CCR5. These viruses typically infect macrophages and primary CD4⁺ lymphocytes, and do not form syncytia in vitro. These viruses are said to be macrophage tropic (M-tropic or R5 strain). After primary HIV-1 infection, viral populations are usually characterized by molecular heterogeneity.

Years after chronic infection is established, strains using CXCR4 emerge in about 50% of infected individuals. CXCR4 strains not only infect primary T lymphocytes but also replicate in T-cell lines and induce syncytia. These viruses are said to be T-cell tropic (T-tropic or X4 strain). This difference in cell tropism correlates with disease progression. During HIV infection, strains isolated from individuals early in the course of their infection are usually M-tropic, while viruses isolated from approximately 50% of individuals with advanced immunodeficiency also include viruses that are T-tropic. This suggests that the ability of the viral envelope to interact with CXCR4 represents an important feature in the pathogenesis of immunodeficiency and the development of full blown acquired immunodeficiency syndrome.

Other HIV coreceptors have also been reported. These coreceptors include, but are not limited to, CCR1, CCR2, CCR3, CCR4, CCR8, CXCR1, CXCR2, CXCR3, CXCR6, GPR15, APJ, CMKLR1, and CX3CR1. CCR1 (chemokine (C—C motif) receptor 1) is a member of the beta chemokine receptor family, which is predicted to have seven transmembrane domains. Chemokines and their receptors mediate signal transductions that are critical for the recruitment of effector immune cells to the site of inflammation. Knockout studies of the mouse CCR1 homolog suggested the roles of this gene in host protection from inflammatory response, and susceptibility to virus and parasite. The CCR1 gene and other chemokine receptor genes including CCR2, CCRL2, CCR3, CCR5 and CCXCR1 form a gene cluster on chromosome 3p. A non-limiting example of human CCR1 sequence is depicted in SEQ ID NO:5.

CCR2 (chemokine (C—C motif) receptor 2; also known as CCR2b) is a receptor for monocyte chemoattractant protein-1, a chemokine which specifically mediates monocyte chemotaxis. Monocyte chemoattractant protein-1 is involved in monocyte infiltration in inflammatory diseases such as rheumatoid arthritis as well as in the inflammatory response against tumors. CCR2 is capable of mediating agonist-dependent calcium mobilization and inhibition of adenylyl cyclase. At least two alternatively spliced CCR2 isoforms have been identified. Exemplary sequences for these two isoforms are depicted in SEQ ID NOs: 6 and 7, respectively.

CCR3 (chemokine (C—C motif) receptor 3) is receptor for C—C type chemokines. It belongs to family 1 of the G protein-coupled receptors. This receptor binds and responds to a variety of chemokines, including eotaxin (CCL11), eotaxin-3 (CCL26), MCP-3 (CCL7), MCP-4 (CCL13), and RANTES (CCL5). It is highly expressed in eosinophils and basophils, and is also detected in TH1 and TH2 cells, as well as in airway epithelial cells. This receptor may contribute to the accumulation and activation of eosinophils and other inflammatory cells in the allergic airway. At least two alternatively spliced transcript variants have been identified for CCR3. Both isoforms encode the same protein. An exemplary sequence for human CCR3 is depicted in SEQ ID NO:8.

CCR4 (chemokine (C—C motif) receptor 4) belongs to the G-protein-coupled receptor family. It is a receptor for the CC chemokine, including MIP-1, RANTES, TARC and MCP-1. CCR4 is expressed with high frequency in adult T-cell leukemia and human T-cell leukemia virus type 1-transformed T cells and in ATL skin lesions. An exemplary human CCR4 sequence is depicted in SEQ ID NO:9.

CCR8 (chemokine (C—C motif) receptor 8) is a member of the beta chemokine receptor family and predicted to have seven transmembrane domains. This receptor protein is preferentially expressed in the thymus. Studies of this receptor and its ligands suggested its role in regulation of monocyte chemotaxis and thymic cell apoptosis. This receptor may contribute to the proper positioning of activated T cells within the antigenic challenge sites and specialized areas of lymphoid tissues. An exemplary human CCR8 sequence is described in SEQ ID NO:10.

CXCR1 (interleukin 8 receptor, alpha; or IL8RA) is a member of the G-protein-coupled receptor family. This protein is a receptor for interleukin 8 (IL8). It binds to IL8 with high affinity, and transduces the signal through a G-protein activated second messenger system. Knockout studies in mice suggested that this protein inhibits embryonic oligodendrocyte precursor migration in developing spinal cord. An exemplary human CXCR1 sequence is illustrated in SEQ ID NO:11.

CXCR2 (interleukin 8 receptor, beta; or IL8RB) is also a member of the G-protein-coupled receptor family. Like CXCR1, this protein is a receptor for interleukin 8 (IL8). CXCR2 binds to chemokine (C—X—C motif) ligand 1 (CXCL1/MGSA), a protein with melanoma growth stimulating activity, and has been shown to be a major component required for serum-dependent melanoma cell growth. CXCR2 mediates neutrophil migration to sites of inflammation. The angiogenic effects of IL8 in intestinal microvascular endothelial cells are found to be mediated by CXCR2. Knockout studies in mice suggested that this receptor controls the positioning of oligodendrocyte precursors in developing spinal cord by arresting their migration. The genes encoding CXCR1 and CXCR2, as well as the IL8RBP gene, form a gene cluster in a region mapped to chromosome 2q33-q36. An exemplary human CXCR2 sequence is depicted in SEQ ID NO:12.

CXCR3 (chemokine (C—X—C motif) receptor 3) is a G protein-coupled receptor with selectivity for three chemokines—namely, IP10 (interferon-g-inducible 10 kDa protein), Mig (monokine induced by interferon-g), and I-TAC (interferon-inducible T cell a-chemoattractant). IP10, Mig and I-TAC belong to the structural subfamily of CXC chemokines, in which a single amino acid residue separates the first two of four highly conserved Cys residues. Binding of chemokines to CD183 induces cellular responses that are involved in leukocyte traffic, including integrin activation, cytoskeletal changes and chemotactic migration. Inhibition by Bordetella pertussis toxin suggests that heterotrimeric G protein of the Gi-subclass couple to CD183. A hallmark of CD183 is its prominent expression in in vitro cultured effector/memory T cells, and in T cells present in many types of inflamed tissues. In addition, IP10, Mig and I-TAC are commonly produced by local cells in inflammatory lesion, suggesting that CD183 and its chemokines participate in the recruitment of inflammatory cells. An exemplary human CXCR3 sequence is provided in SEQ ID NO:13.

CXCR6 (chemokine (C—X—C motif) receptor 6; also known as STRL33) is predominantly localized in colorectal epithelial cells and some scattered stromal cells. It has been reported that HIV-2 isolates from aviremic and viremic individuals commonly use CCR5, GPR15, or CXCR6 as coreceptors, in combination with CD4. A non-limiting example of human CXCR6 sequence is depicted in SEQ ID NO:14.

GPR15 (G protein-coupled receptor 15; also know as BOB) plays a role in HIV gp120 binding to intestinal epithelial cells and gp120-induced cytopathic effects. An exemplary human GRP15 sequence is described in SEQ ID NO:15.

APJ (angiotensin II receptor-like 1 or AGTRL1) mediates effects of angiotensin II. This gene is related to the AGTR1 gene by sequence similarity. It was cloned based on a conserved transmembrane domain found in members of the G protein-coupled receptor gene family. An exemplary human APJ sequence is depicted in SEQ ID NO:16.

CMKLR1 (chemokine-like receptor 1; also known as ChemR23) has been reported to mediate the Resolvin E1 signal to attenuate nuclear factor-κB. A non-limiting example of human CMKLR1 sequence is depicted in SEQ ID NO:17.

CX3CR1 (chemokine (C—X3-C motif) receptor 1) is selectively expressed on various lineages of lymphocytes with high contents of intracellular perforin and granzyme B. The impact of CX3CR1 polymorphisms on HIV-1 pathogenesis and infection progression in children has been reported. A non-limiting example of human CX3CR1 sequence is described in SEQ ID NO:18.

The present invention features modified erythrocytes which comprise CD4 and at least one HIV coreceptor (e.g., 1, 2, 3, 4, 5, 6, 7, 8, 9, 10, or more coreceptors). Preferably, the CD4 or HIV coreceptor proteins employed in the present invention are human proteins (e.g., SEQ ID NOs:1-18). More preferably, the CD4 or HIV coreceptor proteins employed are identical to the corresponding endogenous proteins expressed in the individual being treated. The CD4 or HIV coreceptor proteins can also be modified to reduce or eliminate any potential graft-versus-host and host-versus-graft reactions including the use of endogenous proteins expressed in the individual being treated.

In one embodiment, a modified erythrocyte of the present invention comprises CD4 and at least one HIV coreceptor selected from the group consisting of CCR5, CXCR4, CCR1, CCR2, CCR3, CCR4, CCR8, CXCR1, CXCR2, CXCR3, CXCR6, GPR15, APJ, CMKLR1, and CX3CR1. In another embodiment, a modified erythrocyte of the present invention comprises CD4 and at least two different HIV coreceptors, each of which is selected from the group consisting of CCR5, CXCR4, CCR1, CCR2, CCR3, CCR4, CCR8, CXCR1, CXCR2, CXCR3, CXCR6, GPR15, APJ, CMKLR1, and CX3CR1. In still another embodiment, a modified erythrocyte of the present invention comprises CD4 and at least three different HIV coreceptors, each of which is selected from the group consisting of CCR5, CXCR4, CCR1, CCR2, CCR3, CCR4, CCR8, CXCR1, CXCR2, CXCR3, CXCR6, GPR15, APJ, CMKLR1, and CX3CR1.

In yet another embodiment, a modified erythrocyte of the present invention comprises CD4 and CCR5. The modified erythrocyte may further include one or more HIV coreceptors selected from CXCR4, CCR1, CCR2, CCR3, CCR4, CCR8, CXCR1, CXCR2, CXCR3, CXCR6, GPR15, APJ, CMKLR1, or CX3CR1.

In still yet another embodiment, a modified erythrocyte of the present invention comprises CD4 and CXCR4. The modified erythrocyte may further include one or more HIV coreceptors selected from CCR5, CCR1, CCR2, CCR3, CCR4, CCR8, CXCR1, CXCR2, CXCR3, CXCR6, GPR15, APJ, CMKLR1, or CX3CR1.

In a further embodiment, a modified erythrocyte of the present invention comprises CD4, CCR5, and CXCR4. The modified erythrocyte may further include one or more HIV coreceptors selected from CCR1, CCR2, CCR3, CCR4, CCR8, CXCR1, CXCR2, CXCR3, CXCR6, GPR15, APJ, CMKLR1, or CX3CR1.

In still another embodiment, a modified erythrocyte of the present invention comprises CD4, CCR5, CXCR4, CCR1, CCR2, CCR3, CCR4, CCR8, CXCR1, CXCR2, CXCR3, CXCR6, GPR15, APJ, CMKLR1, and CX3CR1.

The present invention also features modified erythrocytes which comprise one or more HIV coreceptors but not CD4. HIV-1 infection of CD4-negative cells in vitro has been reported. This infection, however, is usually much less efficient than infection of cells that express CD4. It has also been reported that CD4-negative brain astrocytes can be infected by HIV-1 in vivo, particularly in pediatric AIDS patients. This virus appears to utilize CXCR4 to infect CD4-negative cells. Substitution of the V3 loop of the viral gp120 protein with that of an HIV R5 strain can produce viruses capable of CD4-independent infection via CCR5. Certain HIV-2 isolates have also been reported to infect CCR5⁺ or CXCR4⁺ cells without CD4. The efficiency of CD4-independent infection by HIV-2 is often markedly higher than that of HIV-1. Therefore, modified erythrocytes comprising these HIV coreceptors, either in the presence or absence of CD4, can be used to capture and eliminate CD4-independent HIV strains.

In one embodiment, a modified erythrocyte of the present invention comprises CXCR4 but not CD4. The modified erythrocyte may further include one or more coreceptors selected from CCR5, CCR1, CCR2, CCR3, CCR4, CCR8, CXCR1, CXCR2, CXCR3, CXCR6, GPR15, APJ, CMKLR1, or CX3CR1.

In another embodiment, a modified erythrocyte of the present invention comprises CCR5 but not CD4. The modified erythrocyte may further include one or more coreceptors selected from CXCR4, CCR1, CCR2, CCR3, CCR4, CCR8, CXCR1, CXCR2, CXCR3, CXCR6, GPR15, APJ, CMKLR1, or CX3CR1.

In still another embodiment, a modified erythrocyte of the present invention comprises CXCR4 and CCR5 but not CD4. The modified erythrocyte may further include one or more coreceptors selected from CCR1, CCR2, CCR3, CCR4, CCR8, CXCR1, CXCR2, CXCR3, CXCR6, GPR15, APJ, CMKLR1, or CX3CR1

In yet another embodiment, a modified erythrocyte of the present invention comprises at least 1, 2, 3, 4, 5, 6, 7, 8, 9, 10, or more HIV coreceptors, each of which is selected from CXCR4, CCR5, CCR1, CCR2, CCR3, CCR4, CCR8, CXCR1, CXCR2, CXCR3, CXCR6, GPR15, APJ, CMKLR1, or CX3CR1.

The present invention further features modified erythrocytes which comprise CD4 but not other HIV coreceptors. These erythrocytes can compete against CD4⁺ T cells or other cell types for the interaction with HIV virions, thereby reducing the chance of HIV infection of T cells or other cells.

The present invention contemplates the use of any combination of CD4 and/or HIV coreceptors for inclusion in a modified erythrocyte of the present invention. Non-limiting examples of coding sequences for these HIV receptor/coreceptor proteins are depicted in SEQ ID NOs:1-18.

In another aspect, the present invention features the use of functional equivalents of naturally-occurring HIV receptor/coreceptor proteins. These functional equivalents retain their abilities to interact with their respective viral proteins (e.g., gp120), and are capable of mediating HIV entry into host cells. In one embodiment, a functional equivalent of an HIV receptor/coreceptor has the same extracellular domain(s) as the original protein but different transmembrane or intracellular domains. Methods suitable for preparing such a chimeric protein are well known in the art. Any HIV receptor/coreceptor described above can be so modified. The extracellular, transmembrane, or intracellular domains of a naturally-occurring HIV receptor/coreceptor can be determined by using protein structure prediction programs such as TMHMM, or based on the annotations of Entrez or other available databases.

In another embodiment, the functional equivalents are biologically-active variants of HIV receptor/coreceptor proteins. A “variant” is a polypeptide which differs from the original protein by one or more amino acid substitutions, deletions, insertions, or other modifications. These modifications do not significantly change the biological activity of the original protein (e.g., the activity to mediate entry of HIV into host cells). In many cases, a variant retains at least 10%, 20%, 30%, 40%, 50%, 60%, 70%, 80%, 90%, 95%, or 100% of the biological activity of the original protein. The biological activity of a variant can also be higher than that of the original protein. A variant can be naturally-occurring, such as by allelic variation or polymorphism, or deliberately engineered.

The amino acid sequence of a variant is substantially identical to that of the original protein. In many embodiments, a variant shares at least 50%, 60%, 70%, 80%, 85%, 90%, 95%, 99%, or more global sequence identity or similarity with the original protein. Sequence identity or similarity can be determined using various methods known in the art, such as Basic Local Alignment Tool (BLAST), dot matrix analysis, or the dynamic programming method. In one example, the sequence identity or similarity is determined by using the Genetics Computer Group (GCG) programs GAP (Needleman-Wunsch algorithm). Default values assigned by the programs can be employed, e.g., the penalty for opening a gap in one of the sequences is 11 and for extending the gap is 8. Similar amino acids can be defined by the BLOSUM62 substitution matrix. The amino acid sequences of a variant and the original protein can be substantially identical in one or more regions, but divergent in other regions.

Any method known in the art may be used to prepare the biologically-active variants of HIV receptor/coreceptor proteins. For instance, a variant can be prepared from an original protein by adding, deleting, substituting or modifying at least 1, 2, 3, 4, 5, 6, 7, 8, 9, 10, or more amino acid residues without significantly altering the biological activity of the protein. The amino acid residue(s) being substituted can be conservative or non-conservative residue(s). Conservative amino acid substitutions may be introduced into a protein sequence without significantly changing the structure or biological activity of the protein. Conservative amino acid substitutions can be made on the basis of similarity in polarity, charge, solubility, hydrophobicity, hydrophilicity, or the amphipathic nature of the residues. For instance, conservative amino acid substitutions can be made among amino acids with basic side chains, such as lysine (Lys or K), arginine (Arg or R) and histidine (His or H); amino acids with acidic side chains, such as aspartic acid (Asp or D) and glutamic acid (Glu or E); amino acids with uncharged polar side chains, such as asparagine (Asn or N), glutamine (Gln or Q), serine (Ser or S), threonine (Thr or T), and tyrosine (Tyr or Y); or amino acids with nonpolar side chains, such as alanine (Ala or A), glycine (Gly or G), valine (Val or V), leucine (Leu or L), isoleucine (Ile or I), proline (Pro or P), phenylalanine (Phe or F), methionine (Met or M), tryptophan (Trp or W) or cysteine (Cys or C). Examples of commonly used amino acid substitutions are illustrated in Table 1.

Other desired amino acid modifications can also be introduced into an HIV receptor/coreceptor protein. For instance, amino acid modification(s) can be introduced to improve the stability of the protein.

The modified erythrocytes of the present invention can be prepared from erythrocyte precursor cells, such as CD34⁺ hematopoietic progenitor cells. Exemplary procedures suitable for the isolation and culturing of erythrocyte precursor cells are described in Malik et al., Blood, 91:2664-2671 (1998); Hanspal et al., Blood, 84:3494-3504 (1994); Wada et al., Blood, 75:505-511 (1990); and Fibach et al., Blood, 73:100-103 (1989), all of which are incorporated herein by reference. Other methods known in the art can also be used.

Erythrocyte precursor cells can be isolated from peripheral blood, bone marrow, umbilical cord blood, or other suitable sources. Preferably, the donor of the precursor cells is also the recipient of the progeny cells. The precursor cells can also be isolated from donors who have the same blood type as the recipients of the progeny cells. These donors or recipients can be either infected with the virus being treated, or disease-free.

Expression vectors encoding desired HIV receptor/coreceptor proteins (e.g., CD4, CCR5, or CXCR4) can be introduced into erythrocyte precursor cells by transfection, transduction, electroporation, gene gun, or other gene transfer means. Vectors suitable for this purpose include, but are not limited to, viral vectors such as retroviral, lentiviral, adenoviral, adeno-associated viral (AAV), herpes viral, alphavirus, astrovirus, coronavirus, orthomyxovirus, papovavirus, paramyxovirus, parvovirus, picornavirus, poxvirus, or togavirus vectors. Liposomally-encapsulated expression vectors can also be used. An expression vector can be stably or transiently incorporated into the erythrocyte precursor cells. The cells are then cultured under appropriate conditions (e.g., in the presence of macrophages, or high concentrations of EPO in combination with low concentrations of GM-CSF and IL-3) to produce terminally-differentiated erythrocytes that express the desired HIV receptor/coreceptor proteins.

Selection of cells that are transfected or transduced with exogenous sequences is a matter of routine design within the level of ordinary skill in the art. In a non-limiting example, this is achieved by using selectable markers in the exogenous sequences. Markers suitable for this purpose include, but are not limited to, neomycin (G418), hygromycin, puromycin, zeocin, colchine, methotrexate, or methionine sulfoximine resistance genes.

For each expressed HIV receptor/coreceptor protein, an erythrocyte precursor cell can include one or more copies of the coding sequence for that protein. These copies can be carried by the same or different expression vectors. The coding sequences for different HIV receptor/coreceptor proteins can also be carried by the same or different expression vectors. In one example, an erythrocyte precursor cell of the present invention is transfected or transduced with an expression vector which encodes CD4 and an HIV coreceptor selected from CCR5, CXCR4, CCR1, CCR2, CCR3, CCR4, CCR8, CXCR1, CXCR2, CXCR3, CXCR6, GPR15, APJ, CMKLR1 or CX3CR1. In another example, an erythrocyte precursor cell of the present invention is transfected or transduced with an expression vector which encodes CD4 and at least two different HIV coreceptors selected from CCR5, CXCR4, CCR1, CCR2, CCR3, CCR4, CCR8, CXCR1, CXCR2, CXCR3, CXCR6, GPR15, APJ, CMKLR1 or CX3CR1. Any combination of these coreceptors is contemplated by the present invention. In still another example, an erythrocyte precursor cell of the present invention is transfected or transduced with an expression vector which encodes one or more HIV coreceptors but not CD4, where each of the HIV coreceptors is selected from CCR5, CXCR4, CCR1, CCR2, CCR3, CCR4, CCR8, CXCR1, CXCR2, CXCR3, CXCR6, GPR15, APJ, CMKLR1 or CX3CR1.

The present invention further features the use of endogenous HIV receptor/coreceptor genes with modifications in their regulatory sequences. For instance, a viral promoter having high expression activity (e.g., CMV promoter) can be added to or substituted for the promoter of an endogenous HIV receptor/coreceptor gene. Methods suitable for this purpose include homologous recombination or other gene targeting techniques. The introduced viral promoter remains active during the culturing and differentiation of erythrocyte precursor cells, thereby allowing sufficient expression of the endogenous HIV receptor/coreceptor in the terminally-differentiated erythrocytes.

Terminally-differentiated, enucleated erythrocytes can be separated from other cells based on their DNA content. In a non-limiting example, cells are first labeled with a vital DNA dye, such as Hoechst 33342 (Invitrogen Corp.). Hoechst 33342 is a cell-permeant nuclear counterstain that emits blue fluorescence when bound to double-stranded DNA. Undifferentiated precursor cells, macrophages or other nucleated cells in the culture are stained by Hoechst 33342, while enucleated erythrocytes are Hoechst-negative. The Hoechst-positive cells can be separated from enucleated erythrocytes by using fluorescence activated cell sorters or other cell sorting techniques. The Hoechst dye can be removed from the isolated erythrocytes by dialysis or other suitable means.

Erythrocytes thus prepared can be centrifuged and resuspended in appropriate solution (e.g., standard AS-3 solution) for infusion into individuals in need thereof. Preferably, the erythrocytes to be infused have the same ABO type as that of the recipient to minimize the risk of infusion-associated immune reactions. The erythrocytes can also be pretreated to remove blood type-specific antigens or otherwise reduce antigenicities. Methods suitable for this purpose include, but are not limited to, those described in U.S. Patent Application Publication Nos. 20010006772 and 20030207247. In addition to infusion, the modified erythrocytes of the present invention can also be administered via other suitable routes, as appreciated by those of ordinary skill in the art.

The dosage and frequency of the administration can be determined by the attending physician based on various factors such as the severity of disease, the patient's age, sex and diet, the severity of any inflammation, time of administration, and other clinical factors. In one example, an intravenous administration is initiated at a dose which is minimally effective, and the dose is increased over a pre-selected time course until a positive effect is observed. Subsequently, incremental increases in dosage are made limiting to levels that produce a corresponding increase in effect while taking into account any adverse affects that may appear.

Non-limited examples of suitable dosages can range, for example, from 1×10¹⁰ to 1×10¹⁴, from 1×10¹¹ to 1×10¹³, or from 5×10¹¹ to 5×10¹² erythrocytes of the present invention. Specific examples include about 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², or more erythrocytes of the present invention. Each dose of erythrocytes can be administered at intervals such as once daily, once weekly, twice weekly, once monthly, or twice monthly.

The expression level of each HIV receptor or coreceptor protein in the modified erythrocytes can also be adjusted to achieve optimal treatment effects. These can be accomplished by using promoters of different strengths to regulate the expression of the HIV receptor or coreceptor proteins.

Progress of a treatment can be monitored by periodic assessment of disease progression using methods known in the art. For instance, a positive effect can be determined by measuring reduction in viral load, either in plasma or cells (e.g., CD4⁺ cells), increase in T cell or other cell counts (e.g., CD3⁺, CD4⁺, or CD8⁺ cells), or improvement in T cell diversity. Preferably, the modified erythrocytes employed comprise HIV coreceptors that are recognizable or utilized by the HIV strain(s) in the patient being treated.

The modified erythrocytes of the present invention, when administered, bind to plasma HIV and induce the injection of the HIV ribonucleoprotein complex into the cells. Because terminally-differentiated erythrocytes lack nucleic acid synthesis machinery, the entrapped HIV RNA is incapable of being effectively reverse transcribed and is gradually degraded or deactivated within the cells. Any remaining activities of the entrapped HIV content can be eventually destroyed by erythrophagocytosis. In addition, enucleated cells lack nuclei and other machineries necessary for HIV to complete its replication cycle and ultimately manufacture proteins. With no means of replication and no means for escape, HIV components are entrapped in the enucleated cells. Even if the entrapped viral materials escape, these materials are incapable of binding to other cells to initial the fusion process and therefore are not infectious.

The modified erythrocytes of the present invention can be used alone or in combination with other anti-HIV drugs for the treatment or prevention of HIV infections. For instance, the modified erythrocytes of the present invention can be administered with one or more antiretroviral drugs selected from nonnucleoside reverse transcriptase inhibitors (such as delavirdine, Efavirenz, or evirapine); nucleoside reverse transcriptase inhibitors (such as Abacavir, Didanosine, Emtricitabine, Lamivudine, Stavudine, Tenofovir DF, Zalcitabine, or Zidovudine); protease inhibitors (such as Amprenavir, Atazanavir, Fosamprenavir, Indinavir, Lopinavir, Nelfinavir, Ritonavir, or Saquinavir); or fusion inhibitors (such as Enfuvirtide). The modified erythrocytes of the present invention can also be used in conjunction with a HAART regimen.

The above description focuses on modified erythrocytes comprising HIV receptor/coreceptor proteins and methods of using the same to treat or prevent HIV infections. As appreciated by one of ordinary skill in the art, the same methodology can be readily adapted to making modified erythrocytes that comprise receptors for other viruses. These receptors can mediate entry of the corresponding viruses into the modified erythrocytes, thereby preventing the viruses from infecting other cells. The captured virions or their components are degraded or deactivated within the erythrocytes as time elapses, or are eventually destroyed by erythrophagocytosis.

Viruses amenable to the present invention include, but are not limited to, those whose infection involves injection of genetic materials into host cells upon binding to cell surface receptors. Other viruses whose infection is mediated by cell surface receptors can also be treated according to the present invention. Non-limiting examples of these viruses can be selected from Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picornaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). Specific examples of these viruses include human respiratory coronavirus, influenza viruses A-C, hepatitis viruses A to G, and herpes simplex viruses 1-9.

Preferably, a virus being treated circulates in the blood stream, and can be transmitted to a naïve cell through interaction with receptor protein(s) on the cell surface. A modified erythrocyte expressing the receptor protein(s) can be administered to an individual who has contracted or is at risk of contraction of the virus, to reduce the plasma virus titer or the risk of infection. In addition, should the virus face a decreasing ability to access enough host cells per unit of time, this effect correlates with an inability of the virus to perpetuate the infection or perpetuate deleterious effect to the host in question. The viral infection can therefore be suppressed and contained.

The present invention further contemplates the use of other modified cells for the entrapment and elimination of viruses. Non-limiting examples of these cells included T cells, macrophages, neutrophils, natural killer cells, or other leukocytes. These cells can be prepared from hematopoietic progenitor cells or mature cells. Viral receptor proteins or sequences encoding the same can be introduced into hematopoietic progenitor cells or mature non-erythrocyte cells using the methods described above. Hematopoietic progenitor cells that are not modified with exogenous genes can also be employed, provided that the progeny cells derived therefrom comprise the desired endogenous viral receptors. The hematopoietic progenitor cells can be cultured under conditions to allow differentiation into desired cell types. The differentiated cells are then isolated and used for infusion into a patient in need thereof. In many embodiments, the nuclei of the differentiated cells are deactivated before use. Methods suitable for this purpose include radiation, chemical treatment, or other suitable means.

A modified cell of the present invention can also include agents capable of deactivating or destroying the entrapped viral content. Non-limiting examples of suitable agents include anti-viral drugs, proteases, nucleases, antisense molecules, ribozymes, RNAi molecules (e.g., siRNA or shRNA), or other molecules that are toxic or detrimental to the entrapped viral components. These agents can be introduced into a modified cell of the present invention by electroporation, microinjection, gene vectors or other suitable means, as appreciated by one of ordinary skill in the art.

This invention describes cells which circulate or migrate through the body. These cells can be externally created and autologously infused, or, implanted as stem cells which replicate and differentiate, colonize, engraft and produce progeny along the guidelines of this invention. As the cells are intended to circulate, another addition contemplated in this invention touches on each and every type of cell I propose to use. Aside from the provisions of the entirety of this disclosure and the claims, I further provide for the potential to load the cells with a safe compound to further enhance the potential rate of fusion and actual rate of fusion of viruses to the cell. In order to accomplish this, the static charge of the cell, which exists now and is measurable, is intended to be increased. The charge is generated by circulation. The retainage of charge, rate at which a cell may charge can be altered through loading of additional content, or, when the cell is recombinantly produced and cell loading techniques are not to be applied, the expression cassette may include static charge enhancers. As to base elements which in suitable form may be loaded, I include non limiting examples of Iron, Zinc, Cadmium, Selenium and Magnesium as are found naturally in red blood cells. Thus any combination of these metals in suitable for loading in base form to then prove up increases in static production and retention in the cell, as the cells naturally circulate. There are synthetics which could be used to increase the average charge of a cell. Biodegradable polymers, such as certain vinyls, introduced in nano-form, could be considered as static generating candidates. Logically, one merely needs to then calculate the total dosing of these trace minerals or synthetics en masse, so as to add only that which enhances the cell's ability to produce static charge, but does not release enough of the base metal at any time and under any condition, to pose any risk to the health of the subject. Static charge enhancement is very important as the initial contact between any cell and any valid mammalian virus is first induced by the laws of electrostatic attraction and bonding. Thereafter, with many more viruses attached or initially teathered to the cells of this invention via electrostatic bonding, we will then invoke more frequently the stronger bonds, such as hydrophobic and covalent (any form of covalent bonding as applicable to and observed in organic chemistry). In essence, we trip the viral entry mechanism by having the necessary elements in place to do so, then attract more viruses to the location of this motif, with static charge. Through this additional enhancement, aside from all other named enhancements, the cells of this invention can collect more of the intended and targeted viruses and induce more fusion between said cell and said virus during circulation (or equally, the same effect as to any target, such as plasmid or even a molecule we intend to gather). The total static charge can be monitored so the patient does not become a static electricity generator on par with becoming a hazard to electronic equipment and the like. No such level of charge is intended or needed here. It is thus one object of the present invention to provide cells which are fusion capable, fusion enhanced and before fusion can occur, the weak bond of electrostatic between these cells and the target virus, is intended to be enhanced above and beyond other cells found in the body. As a matter of pure logic, or, equally, through mathematic calculation, it is viable to consider the effect a considerable number of red blood cells would have with all aspects of this invention maximized, traversing through a human host without invoking any negative side effect. The cells would first attract more virus to their surface, in the order of 2-100 times more attraction via electrostatic means, and thus would effectively filter virus from tissues and open plasma drawing virus away from other cell types. Thereafter, the fusion enhancements, which are distinguished and different from static bonding, have a greater probability of bonding, fusion and thus drawing in a viral particle from outside the cell to inside the cell. Ideally, electrostatic enhanced cells of this invention can capture incrementally more virus than if the cells were modified in all manners and aspects of this invention minus the electrostatic enhancement(s). In a most preferred embodiment, without inducing any possible negative side effect, I would seek to demonstrate between 2-10000 fold increase in viral capture and fusion efficiency by adding the electrostatic means to the cells which have been prior modified to be fusion enhanced, fusion competent cells targeted to fuse with a given viral class, such as HIV, Hepatitis or other damaging viruses.

Combination uses of this invention yields significantly more effects delivered per cell, with lower cost and reduced effort. Examples include addition of antigen to the cells of this invention, or biomarker, gene chip, protein chip, electronic micro circuit affixed reliably to an otherwise functional cell of this invention. Therein, a therapeutic effect delivered could be two fold, that being viral trap and antigen introduction forming an immune competence builder. Another combination effect could be a preventative effect, in that the cell is a viral trap and the antigen again, forms an advance immune competence to the future presence of the target virus or pathogen. Biomarker and gene/protein chip is a novelty which should be obvious to those of skill. With a reliable biomarker, we know we are observing our own cells in any future removal of said cells from the host. The chip portion could act as a clinical or diagnostic tool, which emerges from the host with other valuable data contained in each cell. Such data can include the titre of virus removed, per cell (efficiency and peak performance, or saturation point if any). Disablement of the internalized viral components could be proven up through introduction of viral component detection, such as RT function, expression, transcription or translation. RBC burst and micro-pipette introduced to an external T Cell line, could quickly demonstrate the virus internalized in the RBC is disabled. A cell, in carrying additional components as defined herein, can form an early reporting and detection system, such as for military use or to simply provide the earliest possible preemptive warning that, for example, HIV has arrived. RBCs traverse the body and in total number, represent a very sensitive component of a system, which could include external detectors which seek a marker provided by the RBC. Therein, a chain reaction effect, synthesized upon the RBC backbone could be strategized and deployed for early warning of the presence or absence of molecular targets. Another effect to consider is the idea that for each molecular target in the body, the RBC or other cell could be equipped to remove said target as a perpetuated cyclic function. eg we make the cells and autologously provide them, or we arrive at a reliable stem cell variant and implant those, or, we arrive at a mechanization which can be internalized into the patient which thereafter, makes the cells needed from cells streamed in from a minor artery and released into a downstream artery or a vein. These combinations are anticipated as stated, and the more utility we can build into these cells, the better the net sum result. The reason for this observation is, it is well anticipated that a very large number of these cells will be manufactured and used en masse. The more useful functions we can provide safely, per cell, the lower the cost and the greater the utility. It is interesting to note, the cells, in performing their functions, can actually warn an early warning system that virus is escaping, for example. Viral escape can be sourced to a mutation or recombination of the virus, or through the host contracting a new strain or variant. Synthetic receptor/coreceptors targeting viruses are not presently known, however, they are claimed herein as formed of xeno-transferred proteins, electronic nano components and static charge enhanced modalities affixed to bilipid membranes. All modalities contained within the 4 corners of this specification are further reclaimed in conjunction with the use of any one or more synthetic variant to produce the same fundamental invention.

The foregoing description of the present invention provides illustration and description, but is not intended to be exhaustive or to limit the invention to the precise one disclosed. Modifications and variations consistent with the above teachings may be acquired from practice of the invention. Thus, it is noted that the scope of the invention is defined by the claims and their equivalents.

TABLE 1
Example of Amino Acid Substitutions

| Original | | More Conservative |
|---|---|---|
| Residues | Exemplary Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, 1,4 Diamino-butyric Acid, Gln, | Arg |
| | Asn | |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine | Leu |

1. An isolated erythrocyte comprising a recombinantly produced receptor protein capable of binding to a virus, wherein said receptor protein comprises an extracellular domain of an HIV coreceptor and further comprises recombinantly produced fusion enhancers or cell loaded fusion enhancers.

2. The erythrocyte of claim 1 wherein said erythrocyte further comprises an extracellular domain of CD4 and fusion enhancers where said fusion enhancer is one of a short residue sequence extracted from a virus, HIV-1 FP23, the 23 N-terminal peptide of the HIV-1 gp 41 protein (AVGIGALFLGFLGAAGSTMGARS).

3. The erythrocyte of claim 1, wherein said erythrocyte further comprises CD4 and fusion enhancer HIV-1 FP23 the 23 N-terminal peptide of the HIV-1 gp 41 protein (AVGIGALFLGFLGAAGSTMGARS).

4. The erythrocyte of claim 1, wherein said erythrocyte comprises a recombinantly produced receptor protein capable of binding to a virus, wherein said receptor protein further comprises CD4, an HIV coreceptor selected from the group consisting of CXCR4, CCR5, CCR1, CCR2, CCR3, CCR4, CCR8, CXCR1, CXCR2, CXCR3, CXCR6, GPR15, APJ, CMKLR1, CX3CR1 and fusion enhancers selected from the group consisting of fusin, actin, cholesterol (rafts or nono-fragments), viral derived fusion peptide, a long viral protein HIV GP120 or HIV GP41, a portion of HIV GP120 or HIV GP41 given as FP23 or the 23 N-terminal peptide of the HIV-1 gp 41 protein (AVGIGALFLGFLGAAGSTMGARS).

5. A method for producing an erythrocyte comprising a recombinantly produced receptor protein capable of binding to a virus wherein said receptor is CD4 and said erythrocyte further comprises an HIV coreceptor and fusion enhancers selected from the group consisting of fusin, actin, cholesterol (rafts or nono-fragments), fusion peptide, a long viral protein HIV GP120 or HIV GP41, or a shorter derivative of the long viral proteins HIV GP120 or GP41 the method comprising the steps of:
isolating a hematopoietic progenitor cell from a subject;
introducing into the hematopoietic progenitor cell an expression vector which encodes said receptor protein, said coreceptor protein and a viral fusion enhancer protein; and
differentiating the hematopoietic progenitor cell into enucleated erythrocytes; and cell loading of fusion enhancers selected from the group consisting of fusin, actin, cholesterol (rafts or nono-fragments), fusion peptide, a long viral protein such as HIV GP120 or HIV GP41, or a shorter derivative of a long viral protein, the 23 N-terminal peptide of the HIV-1 GP 41 protein (AVGIGALFLGFLGAAGSTMGARS) known as HIV-1 FP23.

6. The erythrocyte of claim 1 where said erythrocyte is a cell of a type other than an erythrocyte.

7. The erythrocyte of claim 2 where said erythrocyte is a cell of a type other than an erythrocyte.

8. The erythrocyte of claim 3 where said erythrocyte is a cell of a type other than an erythrocyte.

9. The erythrocyte of claim 4 where said erythrocyte is a cell of a type other than an erythrocyte.

10. The erythrocyte of claim 5 where said erythrocyte is a cell of a type other than an erythrocyte.


SEQUENCE LISTING (18 sequences)

SEQ ID NO: 1 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 2 - protein, 352 aa, Homo sapiens [sequence omitted]

SEQ ID NO: 3 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 4 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 5 - protein, 355 aa, Homo sapiens
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 6 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 7 - protein, 360 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 8 - protein, 355 aa, Homo sapiens
MTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 9 - protein, 360 aa, Homo sapiens
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 10 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 11 - protein, 350 aa, Homo sapiens
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 12 - protein, 360 aa, Homo sapiens
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 13 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 14 - protein, 342 aa, Homo sapiens
MAEHDYHEDYGFSSFNDSSQEEHQDFLQFSKVFLPCMYLVVFVCGLVGNSLVLVISIFYHKLQSLTDVFLVNLPLADLVFVCTLPFWAYAGIHEWVFGQVMCKSLLGIYTINFYTSMLILTCITVDRFIVVVKATKAYNQQAKRMTWGKVTSLLIWVISLLVSLPQIIYGNVFNLDKLICGYHDEAISTVVLATQMTLGFFLPLLTMIVCYSVIIKTLLHAGGFQKHRSLKIIFLVMAVFLLTQMPFNLMKFIRSTHWEYYAMTSFHYTIMVTEAIAYLRACLNPVLYAFVSLKFRKNFWKLVKDIGCLPYLGVSHQWKSSEDNSKTFSASHNVEATSMFQL

SEQ ID NO: 15 - protein, 360 aa, Homo sapiens
MDPEETSVYLDYYYATSPNSDIRETHSHVPYTSVFLPVFYTAVFLTGVLGNLVLMGALHFKPGSRRLIDIFIINLAASDFIFLVTLPLWVDKEASLGLWRTGSFLCKGSSYMISVNMHCSVLLLTCMSVDRYLAIVWPVVSRKFRRTDCAYVVCASIWFISCLLGLPTLLSRELTLIDDKPYCAEKKATPIKLIWSLVALIFTFFVPLLSIVTCYCCIARKLCAHYQQSGKHNKKLKKSIKIIFIVVAAFLVSWLPFNTFKFLAIVSGLRQEHYLPSAILQLGMEVSGPLAFANSCVNPFIYYIFDSYIRRAIVHCLCPCLKNYDFGSSTETSDSHLTKALSTFIHAEDFARRRKRSVSL

SEQ ID NO: 16 - protein, 380 aa, Homo sapiens
MEEGGDFDNYYGADNQSECEYTDWKSSGALIPAIYMLVFLLGTTGNGLVLWTVFRSSREKRRSADIFIASLAVADLTFVVTLPLWATYTYRDYDWPFGTFFCKLSSYLIFVNMYASVFCLTGLSFDRYLAIVRPVANARLRLRVSGAVATAVLWVLAALLAMPVMVLRTTGDLENTTKVQCYMDYSMVATVSSEWAWEVGLGVSSTTVGFVVPFTIMLTCYFFIAQTIAGHFRKERIEGLRKRRRLLSIIVVLVVTFALCWMPYHLVKTLYMLGSLLHWPCDFDLFLMNIFPYCTCISYVNSCLNPFLYAFFDPRFRQACTSMLCCGQSRCAGTSHSSSGEKSASYSSGHSQGPGPNMGKGGEQMHEKSIPYSQETLVVD

SEQ ID NO: 17 - protein, 371 aa, Homo sapiens
MEDEDYNTSISYGDEYPDYLDSIVVLEDLSPLEARVTRIFLVVVYSIVCFLGILGNGLVIIIATFKMKKTVNMVWFLNLAVADFLFNVFLPIHITYAAMDYHWVFGTAMCKISNFLLIHNMFTSVFLLTIISSDRCISVLLPVWSQNHRSVRLAYMACMVIWVLAFFLSSPSLVFRDTANLHGKISCFNNFSLSTPGSSSWPTHSQMDPVGYSRHMVVTVTRFLCGFLVPVLIITACYLTIVCKLQRNRLAKTKKPFKIIVTIIITFFLCWCPYHTLNLLELHHTAMPGSVFSLGLPLATALAIANSCMNPILYVFMGQDFKKFKVALFSRLVNALSEDTGHSSYPSHRSFTKMSSMNERTSMNERETGML

SEQ ID NO: 18 - protein, 355 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL